(19) United States
(12) Patent Application Publication
(10) Pub. No.: US 2009/0234365 A1
(43) Pub. Date: Sep. 17, 2009

(54) DYNAMOMETRIC TOOL FOR MEDICAL USE

(76) Inventor: Silver Gross, La Chaux-de-Fonds (CH)

Correspondence Address: YOUNG & THOMPSON, 209 Madison Street, Suite 500, ALEXANDRIA, VA, 22314, US

(21) Appl. No.: 12/302,369

(22) PCT Filed: May 22, 2007
(86) PCT No.: PCT/EP2007/054906

§ 371 Date: Nov. 25, 2008

(30) Foreign Application Priority Data
May 26, 2006 (CH) 00857/06

(51) Int. Cl. A61B 17/58

(52) U.S. Cl. 606/104

(57) ABSTRACT

A dynamometric tool for medical use includes: a hollow grip (10), an instrument holder (30) designed to be secured in rotation to an instrument configured to co-operate with an object to be screwed, the instrument holder being extended by a shaft (18) pivoting inside the grip, the instrument holder (30) being frictionally connected to the grip via a spring member (34) arranged between the shaft (18) and the grip (10). According to the invention, the spring member (34) includes plural blades (36) elastically deformable in a substantially radial direction.

### TECHNICAL FIELD

The present invention relates to the field of tools for medical use. It more particularly concerns a dynamometric tool intended to tighten or loosen screws or various objects comprising a screw pitch, during a surgical procedure.

It is, in fact, particularly important to avoid applying uncontrolled tightening torques, for example in the case where a plate is fixed on a bone to repair a fracture. If the tightening applied is excessive, this may lead to crushing the bone and further damaging it.

### PRIOR ART

Known in the prior art are dynamometric tools for screwing, and sometimes unscrewing, various objects, in particular screws for fixing reconstructive elements in reparative surgery. As an example of application, plates can be screwed into fractured bones in order to facilitate their repair. A tool of this type comprises:
a grip so that the surgeon can manipulate it, and
an instrument holder designed to be secured in rotation to an instrument configured to cooperate with the object to be screwed.

In certain tools of this type, the holder is extended by a shaft pivoting inside the grip. It is frictionally connected to the grip via a spring member arranged between the shaft and the grip. The friction is provided by two Breguet toothings, i.e. saw-shaped serrated toothings, one being integral with the holder and the other being secured in rotation with the grip. Springs press the two Breguet toothings against each other so as to secure the holder and the grip in rotation. When the tightening torque is greater than the friction imposed between the two Breguet toothings by the springs, these rub against each other and escape each other. The instrument holder is then no longer driven in rotation by the grip. The maximal applicable tightening torque can be adjusted by modulating the pressure exerted by the springs.

The friction created between the toothings is particularly significant and it is necessary, in order to obtain acceptable precision and longevity of the tool, for the Breguet toothings to be metallic. They, as well as the springs, are made in stainless steel, allowing surgical use which is as hygienic as possible. It is, however, necessary to grease the metallic parts in friction, which is not very satisfactory from a sanitary perspective, since a risk of grease flow outside the tool exists during sterilization operations. Moreover, the precision of such a tool is not very satisfactory (+/−10%) and it is necessary to perform calibrations regularly.

Furthermore, when the maximal tightening torque is reached and the toothings escape each other, this causes jumps in the longitudinal direction of the tool, which is not pleasant for the surgeon and can cause him to make a clumsy gestures.

Moreover, the materials used to produce this tool make it heavy and not very practical.

The present invention therefore aims to propose a dynamometric tool free of the abovementioned drawbacks and which, in particular, is precise, light, and easy to manipulate. Particularly, when the maximal tightening torque is reached, no parasitic jumps are felt by the user.

### BRIEF DESCRIPTION OF THE INVENTION

More precisely, the invention concerns a dynamometric tool for medical use comprising:
a hollow grip,
an instrument holder designed to be secured in rotation to an instrument configured to cooperate with an object to be screwed, said instrument holder being extended by a shaft pivoting inside the grip.

The instrument holder is frictionally connected to the grip via a spring member arranged between the shaft and the grip.

According to the invention, the spring member comprises a plurality of blades elastically deformable in a substantially radial direction.

According to one advantageous embodiment, the spring member comprises a plurality of blades arranged primarily in non-radial directions.

### BRIEF DESCRIPTION OF THE DRAWINGS

Other details will appear more clearly upon reading the following description, done in regard to the appended drawings, in which:

FIG. 1 is a longitudinal cross-sectional view of the device according to the invention;

FIG. 2 is a transverse cross-sectional view of the grip, comprising a close-up plane of one area of this part,

FIG. 3 particularly illustrates one embodiment of a spring member particularly adapted for implementation of the tool according to the invention, and

FIGS. 4a and 4b show, in cross-section and top view, respectively, a second embodiment of a spring member.

### DETAILED DESCRIPTION OF THE INVENTION

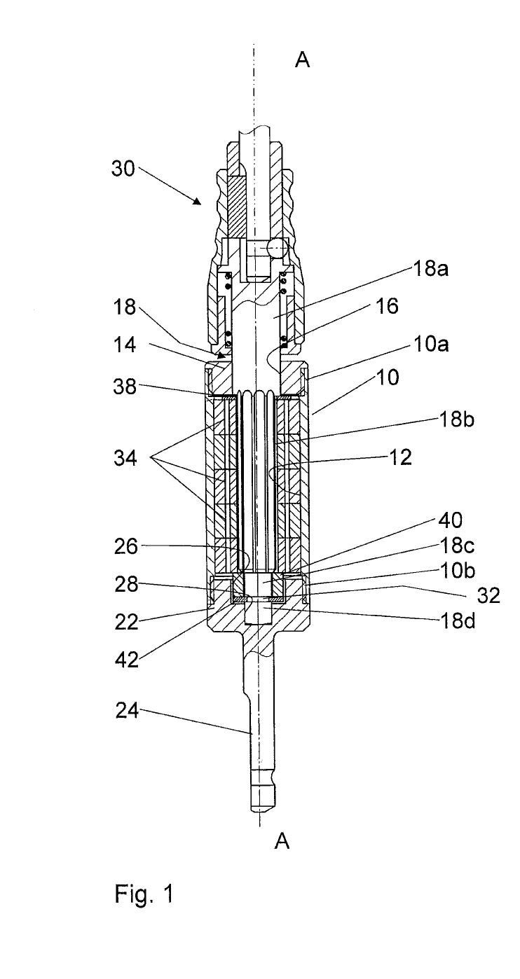

FIG. 1 shows a grip 10, with a longitudinal axis AA. Along this axis, the body 10 is passed through by a cylindrical channel 12. A first end of the grip is provided with a screw pitch 10a to cooperate with a first stopper 14. The latter part comprises a hole 16 intended to allow the passage of a shaft 18 which will be described in more detail below.

The second end of the grip is also provided with a screw thread 10b to cooperate with a second stopper 22. In the illustrated embodiment, this is extended by a rod 24 which can be connected to an apparatus not shown, provided with an engine to cause the rod to rotate. In variation, a sleeve can be fixed around the rod 24 to extend the grip 10 and facilitate manipulation of the tool.

From the inside of the body 10, the walls of the second stopper 22 form a housing 26 whereof the bottom has a cylindrical orifice 28 intended to receive the end of the shaft 18.

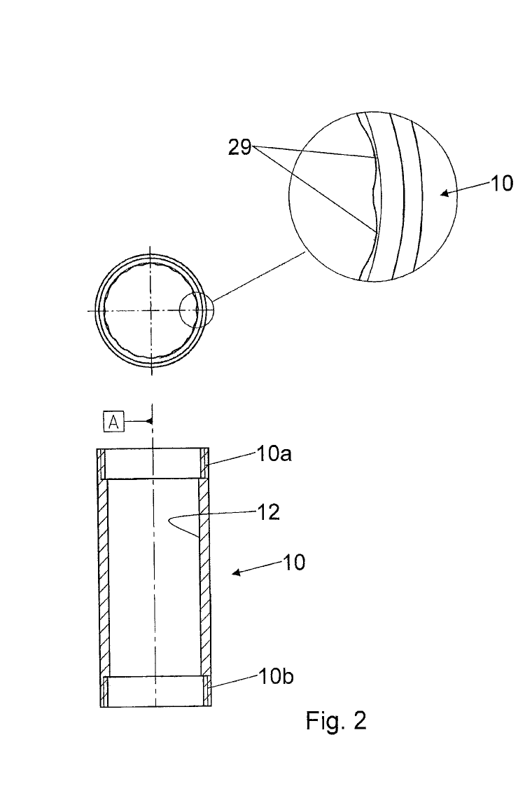

FIG. 2 is a detailed illustration of an embodiment of the structure of the channel 12 whereof the inner wall has a succession of hollows 29, typically with a generally cylindrical shape, oriented along the axis AA. The hollows 19 occupy the entire length of the channel between the two thread pitches 10a and 10b.

In FIG. 1 is also illustrated a holder 30 of the type known by one skilled in the art, of a nature to be secured in rotation to an instrument configured to cooperate with an object to be screwed. The part of the holder providing the connection with the instrument is not in itself part of the invention and will not be described in detail.

The instrument holder 30 is extended by the previously mentioned shaft 18. This is sized so as to be able to go through the hole 16, take a position inside the channel 12 of the grip 10, while its free end takes a position in the housing 26 and in the orifice 28. More precisely, the shaft 18 comprises a first portion 18a adjusted to the dimension of the hole 16. Then, going away from the part of the instrument holder intended to receive an instrument, a second portion 18b intended to be housed in the channel 12. The second portion 18b is structured so as to have a typically star-shaped section, and thus forms a male member able to be connected in rotation with a female member having a corresponding shape. Lastly, the shaft ends with a third portion 18c intended to assume a position in the housing 26 and by a fourth portion 18d adjusted to the dimensions of the orifice 28. These last two portions are separated by an annular groove 32 orthogonal to the longitudinal axis of the shaft.

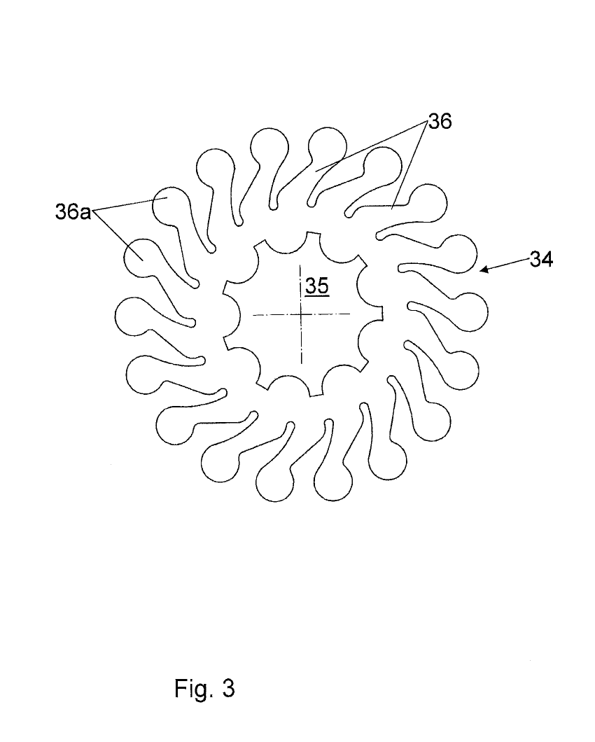

The instrument holder 30 is intended to be mounted pivoting, frictionally, in the channel 12. According to one important aspect of the invention, the friction is provided by at least one spring member 34, an example of which is illustrated in FIG. 3.

This spring member 34 is generally cylindrically shaped and has, in its center, an opening 35 structured so as to have a typically star-shaped section, and thus forms a female member configured to be mounted without play and secured in rotation on the portion 18b of the shaft 18. The proposed star shape is only an example, and other types of slots, structures or lugs can be used, the essential being that these elements are secured in rotation.

The spring member 34 comprises a plurality of blades 36 elastically deformable in a substantially radial direction. These blades 36 are oriented outwardly, primarily along non-radial directions, and end, in one preferred embodiment, with a cylindrical portion 36a substantially orthogonal to the general plane of the spring member.

The spring member 34 is intended to be mounted on the shaft 18, inserted between the latter part and the wall of the channel 12. The cylindrical portions 36a are therefore defined so as to cooperate with the hollows 29 of the channel. Other shapes may be chosen as long as the ends of the blades 36 are able to cooperate with the structures of the wall of the channel 12 to create friction.

In terms of dimensions, one skilled in the art can choose the length, orientation, number and thickness (along a direction perpendicular to the general plane of the spring member) of the blades 36 of the spring member 34 so as to define the friction exerted between the spring member 34 and the grip 10. These different parameters make it possible to determine the value of the maximal applicable tightening torque.

The number of springs 34 arranged on the shaft 18 can also be modulated to adjust the maximal tightening torque. However, one will understand below, in describing the assembly of the tool, that nearly all of the length of the second portion 18b of the shaft must be occupied by the spring members. Thus, to vary the number of spring members arranged on the shaft, it is possible to provide for instrument holders and grips with adapted lengths.

Another advantageous solution consists of replacing one spring member with a wedging device, having a similar structure, but not comprising a contact point with the grip. This device can consist of a spring member from which the cylindrical portions have been removed. A simple bush can also replace a spring member to decrease the maximal tightening torque. Preferably, the spring members are centered on the portion 18b, i.e. the wedging devices are also distributed on both sides of the spring members. One skilled in the art can also provide for spring members 34 having different thicknesses in order to provide other possibilities for adjustment. Any operations to adjust the maximal tightening torque are done in the factory. Preferably, the user cannot, for safety and traceability reasons, modulate the maximal tightening torque value himself.

The spring member is advantageously made in a self-lubricating plastic material resistant to typical sterilization, thermal and radiation treatments. Different tests have made it possible to demonstrate that polymers of the polyether-ether-cetone type (known under the name PEEK) had all of the required characteristics. One may more particularly choose PEEK 151G.

Typically, spring members as described above and made in PEEK make it possible to obtain tightening torque values in the vicinity of several N.m, typically between 1 and 10 N.m.

Thus, in order to assemble the tool as illustrated in FIG. 1, the following operations are performed, preferably in a factory by an operator. The first stopper 14 is first firmly fixed on the grip and the shaft of the instrument holder is introduced into the hole 16. Then, a ring 38 is assembled on the shaft 18 so as to define a precise support surface for the spring members 34, the chosen number of which is inserted between the shaft 18 and the channel 12. The spring members are therefore secured in rotation with the shaft 18 and in contact with the wall of the channel 12.

Then, a wedge 40 is arranged on the shaft 18, at the level of the third portion 18c. This wedge 40 must be supported on the last spring member arranged in the channel 12 and not on any threshold located between the second and third portions. The wedge 40 is dimensioned to occupy exactly the space between the last spring and the groove 32. A blocking member 42 is then arranged in this groove 32 to axially position the elements already put into place. Lastly, the second stopper 22 and the rod 24 are screwed to the end of the grip 10 to ensure the maintenance of the assembly thus formed.

The ring 36 on one hand, the wedge 40 and the blocking member 42 on the other, make it possible to longitudinally position the grip in relation to the instrument holder and to greatly limit the friction between the stoppers and the spring members in order to be able to perfectly control the adjustment of the maximal tightening torque.

One will note that the tightening torque for the stoppers must be greater than the determined maximal tightening torque. Preferably, the stoppers can be welded to the grip 10.

Thus is obtained a tool whereof the adjustment of the maximal tightening torque can be particularly precise, with an improved lifespan. Tests have made it possible to show that the precision achieved was in the vicinity of 3% for 10,000 releases. A release is defined as being the moment when the grip is separated from the instrument holder, going from a first to a second relative position of one of these elements in relation to the other. Moreover, thanks to the radial deformation of the spring members, there is no parasitic movement to lament when the maximal tightening torque is achieved. The tool does not need to be lubricated and can be sealed, avoiding any risk of contaminating the patient.

Moreover, thanks to the orientation of the blades, the maximal friction exerted between the spring members and the wall of the channel is different in the direction of screwing and the unscrewing direction. The orientation of the blades is chosen such that a same tool can be used, without modifying the adjustment of the maximal tightening torque, to immediately unscrew the object which has just been screwed.

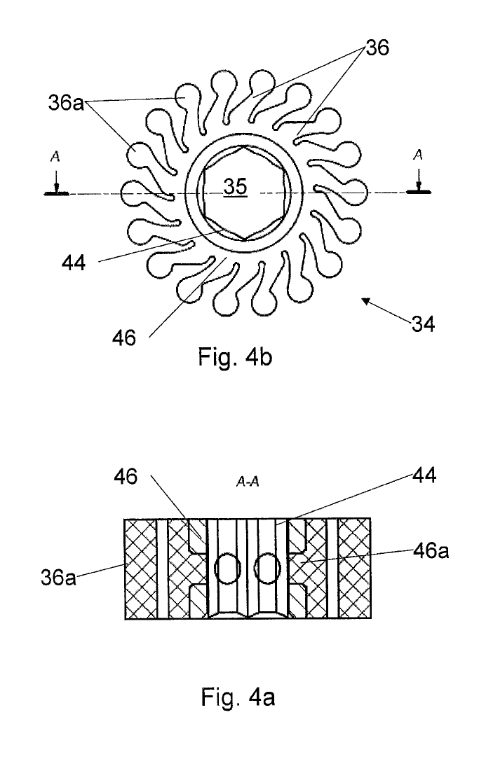

For significant torques, a spring member according to a second embodiment is proposed. In order to improve the connection with the shaft, the spring member as illustrated in FIG. 4 comprises a hub 44 made in a material not elastically deformable, for example of the metallic type, such as a stainless steel. As above, the hub has an opening 35 structured so as to form a female member configured to be mounted without play and secured in rotation on the portion 18b of the shaft 18.

The spring member also comprises a ring 46 supporting the blades 36. This ring is made in one of the materials proposed above for the spring member. The ring is arranged around the hub, securely.

The ring can be molded by casting on the hub. To do this, the hub advantageously has channels going through it transversely and leading into the opening 35. These channels are used to inject the plastic material making it possible to realize the ring. As a result of this production method, the ring has rods 46a housed without play in the channels and which provide an excellent, particularly rigid connection, between the hub and the ring. Moreover, the injection step thus done does not leave any trace on the upper and lower parts of the spring members, which avoids any retouching operation.

The description above was provided solely as an illustration of the invention and does not limit it. Thus, without going outside the scope of the invention, one skilled in the art could also choose to arrange spring members still on the shaft of the instrument holder, but such that the spring members are secured in rotation with the grip and rub on the structures comprised by the shaft. Thus, in such an alternative, the channel can typically have a polygonal section with which the spring members cooperate. The latter parts have blades which are oriented inwardly and which cooperate with structures having an adapted shape arranged in the shaft. A spring member in two parts could be very well adapted to this embodiment.

1-11. (canceled)

12. A dynamometric tool for medical use comprising:
a hollow grip,
an instrument holder to be secured in rotation to an instrument configured to cooperate with an object to be screwed, said holder being extended by a shaft pivoting inside the body,
said instrument holder being frictionally connected to the grip via a spring member arranged between said shaft and said grip, wherein said spring member comprises a plurality of blades elastically deformable along a substantially radial direction.

13. The tool of claim 12, wherein the spring member comprises a plurality of blades arranged primarily along non-radial directions.

14. The tool of claim 12, wherein said spring member comprises a hub made in a material not elastically deformable and a ring supporting the blades and mounted securely around the hub.

15. The tool of claim 13, wherein said spring member comprises a hub made in a material not elastically deformable and a ring supporting the blades and mounted securely around the hub.

16. The tool of claim 14, wherein said hub is provided with channels going through it transversely and wherein said ring has rods lodged without play in the channels.

17. The tool of claim 15, wherein said hub is provided with channels going through it transversely and wherein said ring has rods lodged without play in the channels.

18. The tool of claim 12, wherein the inside of said grip has a cylindrical shape and wherein said spring member is secured in rotation with said instrument holder and wherein said blades are oriented outwardly.

19. The tool of claim 12, wherein the inner surface of said grip has a structure configured to cooperate with the ends of the blades of the spring member.

20. The tool of claim 19, wherein the blades of the spring member end with a cylindrical portion intended to be substantially parallel to said shaft and wherein the inner surface of said grip has a succession of generally cylindrical-shaped hollows, intended to be substantially parallel to said shaft and configured to cooperate with said cylindrical portions.

21. The tool of claim 12, wherein said spring member is integral in rotation with said grip and wherein said blades are oriented inwardly.

22. The tool according to claim 21, wherein the surface of said shaft has a structure configured to cooperate with the ends of said blades.

23. The tool according to claim 22, wherein the blades of the spring member end with a cylindrical portion intended to be substantially parallel to said shaft and wherein the surface of said shaft has a succession of generally cylindrical-shaped hollows, intended to be substantially parallel to said shaft and configured to cooperate with the cylindrical portions of the spring member.

24. The tool of claim 12, wherein said spring member is made in PEEK.

25. The tool of claim 13, wherein said spring member is integral in rotation with said grip and wherein said blades are oriented inwardly.
